(19) United States
(12) Patent Application Publication
(10) Pub. No.: US 2003/0167132 A1
(43) Pub. Date: Sep. 4, 2003

(54) Method and computer software product for predicting polyadenylation sites

(75) Inventors: Simon Cawley, Oakland, CA (US); Raymond Wheeler, Berkeley, CA (US); David Kulp, Albany, CA (US); Alan Williams, Albany, CA (US); Brant Wong, Walnut Creek, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: AFFYMETRIX, INC., Santa Clara, CA

(21) Appl. No.: 10/028,416

(22) Filed: Dec. 21, 2001

(51) Int. Cl. G06F 19/00; G01N 33/48; G01N 33/50

(52) U.S. Cl. 702/20

(57) ABSTRACT

In one embodiment of the invention, computerized methods are provided for detecting polyadenylation sites and polyadenylation signals.

### RELATED APPLICATIONS

This application is related to U.S. patent application Ser. No. 09/721,042, filed on Nov. 21, 2000, entitled “Methods and Computer Software Products for Predicting Nucleic Acid Hybridization Affinity”; U.S. patent application Ser. No. 09/718,295, filed on Nov. 21, 2000, entitled “Methods and Computer Software Products for Selecting Nucleic Acid Probes”; U.S. patent application Ser. No. 09/745,965, filed on Dec. 21, 2000, entitled “Methods For Selecting Nucleic Acid Probes”; U.S. patent application Ser. No. 10/006,174, filed on Dec. 4, 2001, and U.S. patent application Ser. No. ______, attorney Docket No. 3440, filed on Dec. 21, 2001, and U.S. patent application Ser. No. ______, attorney docket number 3441, filed on Dec. 21, 2001. All the cited applications are incorporated herein by reference in their entireties for all purposes.

### BACKGROUND OF THE INVENTION

This invention is related to bioinformatics and biological data analysis. Specifically, this invention provides methods, computer software products and systems for determining the orientation of biological sequences. In some embodiments, the methods, computer software products and systems are used for designing nucleic acid probe arrays.

Polyadenylation occurs at the 3′-end of most mRNA molecules. Polyadenylation stabilizes mRNAs and may regulate translation. Adenines (up to approximately 250) are added by a poly A polymerase after cleavage of the 3′-end of the pre-mRNA near a conserved sequence (AAUAAA, or a close variant thereof). Proteins bind the poly(A) tail influencing the rate of degradation of the RNA and its translation.

Several methods for gene expression monitoring employs the poly-adenylation region for reverse transcription using Poly d(T) primers. Because of inefficiency in the reverse transcription reaction, such assays are often 3′ biased, i.e., the labeled targets tend to represent more at the 3′ end of the transcripts. Hybridization probes, therefore, are often selected against the 3′ region of the target transcripts, close to the polyadenylation sites.

### SUMMARY OF THE INVENTION

In one aspect of the invention, methods are provided for the prediction of polyadenylation sites and signals in EST or RNA sequences. Methods are also provided for scanning genomic sequence for polyadenylation signals. Typically, the EST or RNA sequences are scanned for potential adenylation sites. When a polyadenylation site is discovered, the neighboring sequences in EST or RNA sequences or their corresponding genomic sequences are scanned for potential polyadenylation signals. In some embodiments, the site-prediction method also computes a probability of the site, and the signal-prediction method computes a signal probability, conditional on the site being valid. Taking the product of the two yields a summary probability of the site-signal pair.

Where there is genomic sequence available, an extra check may be performed to detect possible cases of internally or falsely-primed EST sequences. If the region 3′ of the predicted polyadenylation site is found to align well to the genome the sequence is flagged as a possible case of internal or false priming.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic showing an exemplary computer system suitable for executing some embodiments of the software of the invention.

FIG. 2 is a schematic showing the architecture of the exemplary computer system of FIG. 1.

FIG. 3 shows an exemplary computer network system suitable for executing some embodiments of the software of the invention.

FIG. 4 shows an exemplary computerized process for predicting polyadenylation sites and signals.

### DETAILED DESCRIPTION

Reference will now be made in detail to the exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Throughout this disclosure, various aspects of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention will employ, unless otherwise indicated, conventional techniques of bioinformatics, computer sciences, immunology, biochemistry, chemistry, molecular biology, microbiology, cell biology, genomics and recombinant DNA, which are within the skill of the art. See, e.g., Setubal and Meidanis, et al., 1997, Introduction to Computational Molecular Biology, PWS Publishing Company, Boston; Human Genome Mapping Project Resource Centre (Cambridge), 1998, Guide to Human Genome Computing, 2nd Edition, Martin J. Biship (Editor), Academic Press, San Diego; Salzberg, Searles, Kasif, (Editors), 1998, Computational Methods in Molecular Biology, Elsevier, Amsterdam; Matthews, PLANT VIROLOGY, 3^{rd} edition (1991); Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987))

System for Sequence Annotation and for Nucleic Acid Probe Array Design

One of skill in the art would appreciate that many computer systems are suitable for carrying out the methods of the invention. Computer software according to the embodiments of the invention can be executed in a wide variety of computer systems.

For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both are incorporated herein by reference in their entireties for all purposes.

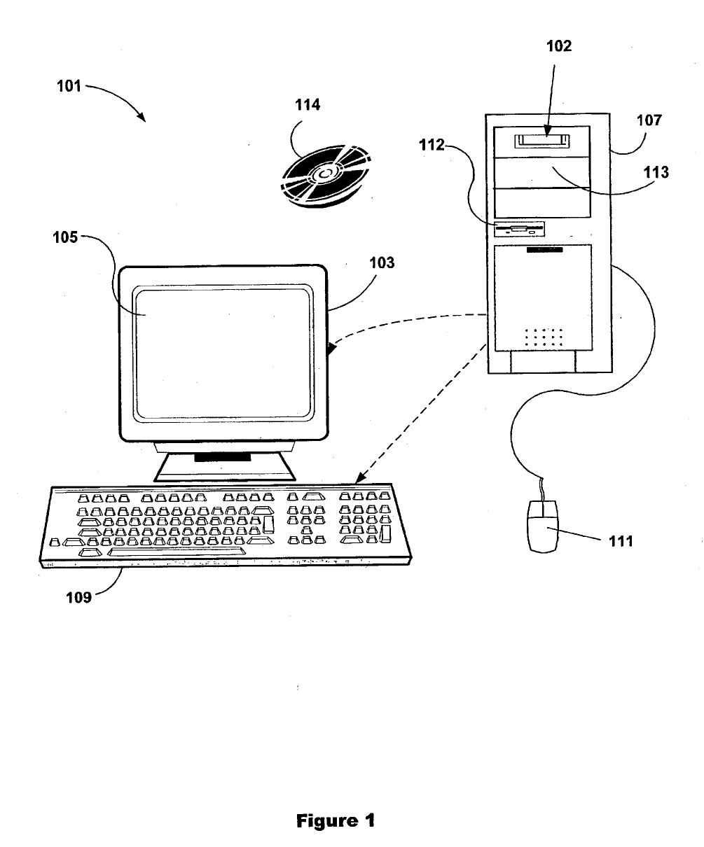

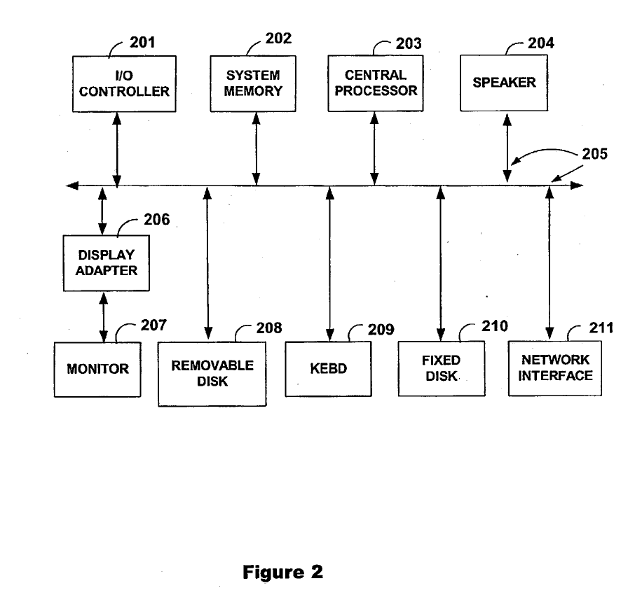

FIG. 1 illustrates an example of a computer system that may be used to execute the software of an embodiment of the invention. FIG. 1 shows a computer system 101 that includes a display 103, screen 105, cabinet 107, keyboard 109, and mouse 11. Mouse 111 may have one or more buttons for interacting with a graphic user interface. Cabinet 107 houses a floppy drive 112, CD-ROM or DVD-ROM drive 102, system memory and a hard drive (113) (see also FIG. 2) which may be utilized to store and retrieve software programs incorporating computer code that implements the invention, data for use with the invention and the like. Although a CD 114 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

FIG. 2 shows a system block diagram of computer system 101 used to execute the software of an embodiment of the invention. As in FIG. 1, computer system 101 includes monitor 201, and keyboard 209. Computer system 101 further includes subsystems such as a central processor 203 (such as a Pentium™ III processor from Intel), system memory 202, fixed storage 210 (e.g., hard drive), removable storage 208 (e.g., floppy or CD-ROM), display adapter 206, speakers 204, and network interface 211. Other computer systems suitable for use with the invention may include additional or fewer subsystems. For example, another computer system may include more than one processor 203 or a cache memory. Computer systems suitable for use with the invention may also be embedded in a measurement instrument.

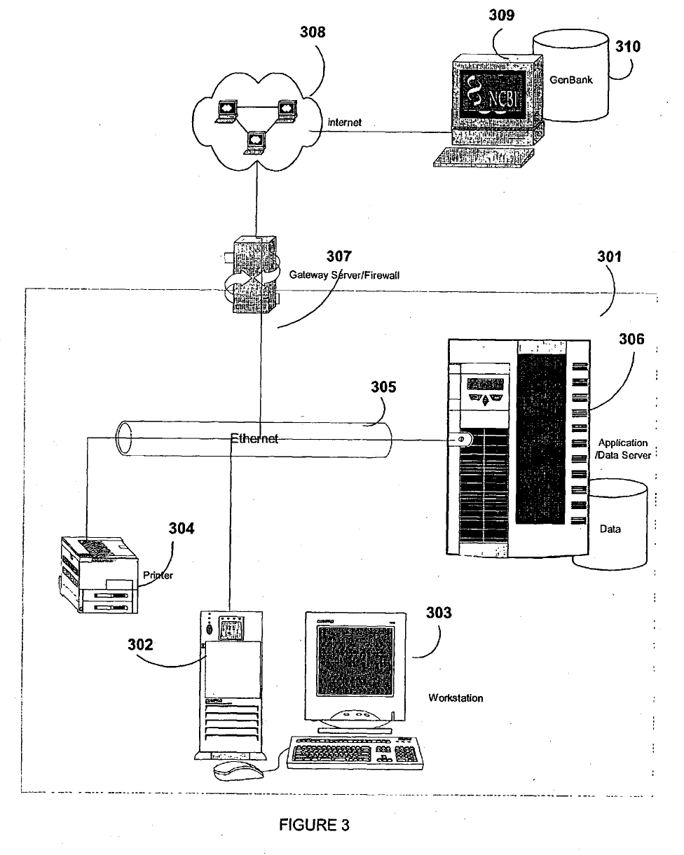

FIG. 3 shows an exemplary computer network that is suitable for executing the computer software of the invention. A computer workstation 302 is connected with the application/data server(s) through a local area network (LAN) 301, such as an Ethernet 305. A printer 304 may be connected directly to the workstation or to the Ethernet 305. The LAN may be connected to a wide area network (WAN), such as the Internet 308, via a gateway server 307 which may also serve as a firewall between the WAN 308 and the LAN 305. In some embodiments, the workstation may communicate with outside data sources, such as the National Biotechnology Information Center, through the Internet. Various protocols, such as FTP and HTTP, may be used for data communication between the workstation and the outside data sources. Outside genetic data sources, such as the GenBank 310, are well known to those skilled in the art. An overview of GenBank and the National Center for Biotechnology information (NCBI) can be found in the web site of NCBI (“www.ncbi.nlm.nih.gov”).

Computer software products of the invention typically include computer readable medium having computer-executable instructions for performing the logic steps of the methods of the invention. Suitable computer readable medium include floppy disk, CD-ROM/DVD/DVD-ROM, hard-disk drive, flash memory, ROM/RAM, magnetic tapes and etc. The computer executable instructions may be written in any suitable computer language or combination of several languages. Suitable computer languages include C/C++ (such as Visual C/C++), C#, Java, Basic (such as Visual Basic), SQL, Fortran, SAS and Perl.

Nucleic Acid Probe Arrays

The methods, computer software and systems of the invention are particularly useful for designing high density nucleic acid probe arrays.

High density nucleic acid probe arrays, also referred to as “DNA Microarrays,” have become a method of choice for monitoring the expression of a large number of genes and for detecting sequence variations, mutations and polymorphism. As used herein, “nucleic acids” may include any polymer or oligomer of nucleosides or nucleotides (polynucleotides or oligonucleotidies), which include pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982) and L. Stryer, BIOCHEMISTRY, 4^{th} Ed. (March 1995), both incorporated by reference. “Nucleic acids” may include any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“A target molecule” refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51, which is incorporated herein by reference for all purposes. For example, if transcripts of genes are the interest of an experiment, the target molecules would be the transcripts. Other examples include protein fragments, small molecules, etc. “Target nucleic acid” refers to a nucleic acid (often derived from a biological sample) of interest. Frequently, a target molecule is detected using one or more probes. As used herein, a “probe” is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that these are illustrative embodiments that are not to limit the invention in any way.

In some embodiments, probes may be immobilized on substrates to create an array. An “array” may comprise a solid support with peptide or nucleic acid or other molecular probes attached to the support. Arrays typically comprise a plurality of different nucleic acids or peptide probes that are coupled to a surface of a substrate in different, known locations. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501, which disclose methods of forming vast arrays of peptides, oligonucleotides and other molecules using, for example, light-directed synthesis techniques. See also, Fodor, et al., Science, 251, 767-77 (1991). These procedures for synthesis of polymer arrays are now referred to as VLSIPS™ procedures.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743 and 6,140,044, all of which are incorporated by reference in their entireties for all purposes.

Microarray can be used in a variety of ways. An exemplary microarray contains nucleic acids and is used to analyze nucleic acid samples. Typically, a nucleic acid sample is prepared from appropriate source and labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine fluorescence intensity distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells (see, GATC™ software specification). The probes in a cell are designed to have the same sequence (i.e., each cell is a probe area). A CEL file contains the statistics of a cell, e.g., the 75th percentile and standard deviation of intensities of pixels in a cell. The 50, 60, 70, 75 or 80th percentile of pixel intensity of a cell is often used as the intensity of the cell.

Nucleic acid probe arrays have found wide applications in gene expression monitoring, genotyping and mutation detection. For example, massive parallel gene expression monitoring methods using nucleic acid array technology have been developed to monitor the expression of a large number of genes (e.g., U.S. Pat. Nos. 5,871,928, 5,800,992 and 6,040,138; de Saizieu et al., 1998, Bacteria Transcript Imaging by Hybridization of total RNA to Oligonucleotide Arrays, NATURE BIOTECHNOLOGY, 16:45-48; Wodicka et al., 1997, Genome-wide Expression Monitoring in Saccharomyces cerevisiae, NATURE BIOTECHNOLOGY 15:1359-1367; Lockhart et al., 1996, Expression Monitoring by Hybridization to High Density Oligonucleotide Arrays. NATURE BIOTECHNOLOGY 14:1675-1680; Lander, 1999, Array of Hope, NATURE-GENETICS, 21(suppl.), at 3, all incorporated herein by reference for all purposes). Hybridization-based methodologies for high throughput mutational analysis using high-density oligonucleotide arrays (DNA chips) have been developed, see Hacia et al., 1996, Detection of heterozygous mutations in BRCA1 using high density oligonucleotide arrays and two-color fluorescence analysis. Nat. Genet. 14:441-447, Hacia et al., New approaches to BRCA1 mutation detection, Breast Disease 10:45-59 and Ramsey 1998, DNA chips: State-of-Art, Nat Biotechnol. 16:40-44, all incorporated herein by reference for all purposes). Oligonucleotide arrays have been used to screen for sequence variations in, for example, the CFTR gene (U.S. Pat. No. 6,027,880, Cronin et al., 1996, Cystic fibrosis mutation detection by hybridization to light-generated DNA probe arrays. Hum. Mut. 7:244-255, both incorporated by reference in their entireties), the human immunodeficiency virus (HV-1) reverse transcriptase and protease genes (U.S. Pat. No. 5,862,242 and Kozal et al., 1996, Extensive polymorphisms observed in HIV-1 clade B protease gene using high density oligonucleotide arrays. Nature Med. 1:735-759, both incorporated herein by reference for all purposes), the mitochondrial genome (Chee et al., 1996, Accessing genetic information with high density DNA arrays. Science 274:610-614) and the BRCA1 gene (U.S. Pat. No. 6,013,449, incorporated herein by reference for all purposes).

Methods for signal detection and processing of intensity data are additionally disclosed in, for example, U.S. Pat. Nos. 5,445,934, 547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996 and 6,136,269, all of which are incorporated by reference in their entireties for all purposes.

Nucleic Acid Probe Array Design Process

In some embodiments, a nucleic acid probe array design process involves selecting the target sequences and selecting probes. For example, if the probe array is designed to detect the expression of genes at the transcript level. The target sequences are typically transcript sequences. Selection of the target sequence may involve the characterization of the target sequence based upon available information. For example, expressed sequence tags information needs to be assembled and annotated.

After target sequences are identified, probes for detecting the target sequences can be selected. The probe sequences and layout information are then translated to photolithographic masks, commands for controlling ink-jet directed synthesis, or soft lithographic synthesis process.

Prediction of Polyadenylation Sites

In one aspect of the invention, methods and computer software is provided to scan EST or RNA sequences and predict the location of polyadenylation sites and signals, taking advantage of alignments to genomic sequence where available.

Most eukaryotes pre-mRNA contain long 3′ untranslated regions (UTRs) of up to several hundreds of nucleotides, and undergoing cleavage and polyadenylation at one or several polyadenylation sites (PAS). Poly A sites are typically defined by a hexameric polyadenylation signal (AAUAA or a close variant thereof), located ˜15 bases upstream of the cleavage site and sometimes, a GU rich element located 20-40 bases down stream of the site (see, e.g., Prodfoot, 1991, “Poly(A) signals” Cell 64: 671-674; Colgan and Manley, 1997, “Mechanism and regulation of mRNA polyadenylation” Gene & Dev. 11:2755-2766, both incorporated herein by reference for all purposes.

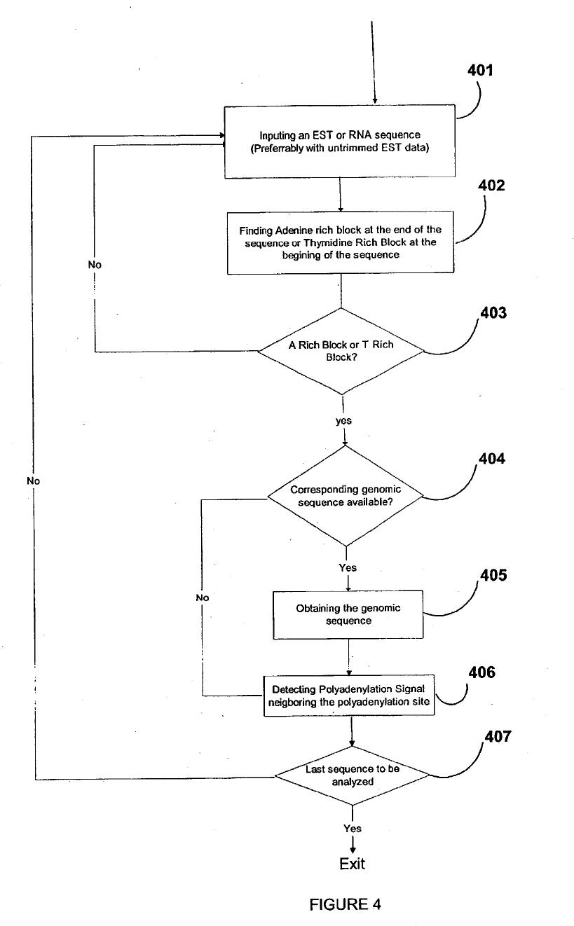

Exemplary methods include two stages (FIG. 4). In the first stage, the EST or RNA is scanned for polyadenylation sites, and if any are found a second pass is made over the sequence to detect polyadenylation signals associated with the site. If there is an alignment to genomic sequence available the genomic is scanned in place of the EST or RNA, as it will usually be of higher quality, having fewer in the way of sequencing errors due to being a consensus of a multitude of sequences.

Detection of polyadenylation sites. Polyadenylation tails are searched at the extrema of the sequence by scanning for a thymine-rich block at the beginning of the sequence or for an adenine-rich block at the end of the sequence. An adenine-rich block is defined as a consecutive run of at least 8, 9, 10, 12 Adenines located within the last 30, 40, 50, 60 bases of the sequence, similarly a thymine-rich block is a run of at least 8, 9, 10, 12 thymines located within the first 30, 40, 50, 60 bases of the sequence. In exemplary embodiments, the methods of the invention use untrimmed sequences, such as the untrimmed EST sequences from Washington University. EST sequences are typically trimmed to remove their polyA/polyT tail before submission to public or privately accessible databases, such as the Unigene. Because the more of the polyadenylation tail occurring in the sequence, the easier it is to find, the method of the invention prefers the input of untrimmed EST sequence data with the poly A/poly T tract intact.

One of skill in the art would appreciate that methods of the invention are not limited to any particular method for scanning a sequence to find adenine or thymine rich region. In an exemplary embodiment for searching an adenine-rich region at the end of the sequence, the sequences are searched first for a block of at least 10 consecutive adenines ocuring within the last 50 bases. If such a run of adenines is found it is extended as far as possible.

Let n_{A} be the number of adenines in the block, and let n_{R} be the number of bases after the block of adenines to the end of the sequence (which may be zero). The location of the polyadenylation site is by convention taken to be the coordinate of the most 3′ base before the adenine block. In one embodiment, a heuristic score is assigned to a detected site according to the formula: n_{A}/(n_{A}+0.5*(max(n_{R}−20,0))). The purpose of the term max(n_{R}−20,0) is to allow for some non-adenine at the extreme of the sequence before penalizing, preferrably 20 bases, possibly 5, 10, 15, 25, or 30 bases. This heuristic score will always be in the range [0,1] and will increase with larger blocks of adenine.

Detection of polyadenylation signals. If a polyadenylation site is found in the EST or RNA, a neighborhood of 50 bases upstream is searched for polyadenylation signals. A polyadenulation signal is typically a hexamer with the sequence AAUAAA (or AATAAA) or a slight variant thereof. In some embodiments, the information used for such detection includes the presence of the typical polyadenylation signal hexamer, its distance from the polyadenylation site and the expected background untranslated region sequence content.

The sequence scanned for the polyadenylation signal is the EST or mRNA sequence, unless there is genomic sequence available in which case that is used instead. The sequence to be scanned can be represented by x=(x₁,x₂, . . . x_{N}) where x_{N} is the 3′-most base before the polyadenylation site. The methods include a step of searching for a polyadenylation signal hexamer, whose position h can be denoted by the coordinate of its 3′-most base. The strategy in some embodiments is to scan through the sequence, at each point evaluating the probability Pr(h=k|x) for k=6,7, . . . ,N. In other words the probability that there is a polyadenylation signal hexamer at position k, conditional on the observed sequence x and on the polyadenylation site just after x_{N}. According to Bayes rule, Pr(h=k|x)=Pr(x|h=k) Pr(h=k)/Pr(x).

In some instances, it can be assumed that the sequence in the candidate hexamer ending at k is independent of the rest of the sequence. This assumption leads to the cancellation of the probabilities involving sequence outside of the hexamer in the numerator and denominator of the equation above, so that we need only consider the candidate hexamer and the equation becomes: Pr(h=k|x)=Pr(xₖ₋₅, . . . , xₖ|h=k) Pr(h=k)/Pr(xₖ₋₅, . . . , xₖ). Pr(h=k) is the probability that the polyadenylation hexamer is located at position k in the sequence, at a distance (N−k) from the polyadenylation site. A gamma function can be used to produce a density which places the majority of its weight on the positions located 5 to 25 bases distant from the polyadenylation site, resulting in a distribution of similar shape as suggested by the data in Beaudoing et al, 2000, “Patterns of variant polyadenylation signal usage in human genes,” Genome Res. 10(7):1001-10. Pr(xₖ₋₅, . . . ,xₖ|h=k) is the probability of observing the hexamer (xₖ₋₅, . . . ,xₖ) given that it is a polyadenylation signal. For human sequences, The positive probability to the following 16 hexamers is assigned according to their observed frequencies in the study conducted by Beaudoing et al (2000):

AATAAA 3286

ATTAAA 843

AGTAAA 156

TATAAA 180

CATAAA 76

GATAAA 72

AATATA 96

AATACA 70

AATAGA 43

AAAAAG 49

ACTAAA 36

AAGAAA 62

AATGAA 49

TTTAAA 69

AAAACA 29

GGGGCT 22

The final term is Pr(xₖ₋₅, . . . ,xₖ)=Pr(xₖ₋₅, . . . ,xₖ|h=k) Pr(h=k)+Pr(xₖ₋₅, . . . ,xₖ|h≠k) Pr(h≠k). The computation of the first two terms is described above, and the last one is simply 1−Pr(h=k). The remaining term Pr(xₖ₋₅, . . . ,xₖ|h≠k) is the probability of observing the hexamer given that it is not from a polyadenylation signal. It can be thought of as the background probability of the hexamer. A second-order Markov model trained on data collected from human 3′ UTRs can be used to model this. One of skill in the art would appreciate that methods of the invention are not limited to any particular method for modeling the 3′UTR data, and that there are many well-established alternatives to using a Markov model. Using a second order Markov model, Pr(xₖ₋₅, . . . ,xₖ|h≠k) can be expressed as Pr(xₖ₋₅) Pr(xₖ₋₄|xₖ₋₅) Pr(xₖ₋₃|xₖ₋₅,xₖ₋₄) Pr(xₖ₋₂|xₖ₋₄,xₖ₋₃) Pr(xₖ₋₁|xₖ₋₃,xₖ₋₂) Pr(xₖ|xₖ₋₂,xₖ₋₁), where the first term is a zero-order Markovian probability, the second is a first-order Markovian probability and the remaining four terms are second-order Markovian probabilities. Maximum-likelihood estimation of these Markovian probabilities can be performed in the following manner. For a k^{th}-order Markov model, the probability of base b following a word w of length k is estimated by the frequency of the concatenated word (wb) divided by the frequency of the word w, where frequencies are computed from the training dataset of 3′UTR sequences. For the case k=0 (a zero-order Markovian model) the probability of base b is estimated simply by its frequency in the dataset divided by the size of the dataset.

In another aspect of the invention, methods are provided for selecting 3′ biased probes for gene expression monitoring. The probes are particularly useful for experiments where the transcript samples are processed such that the derived nucleic acids for hybridization are rich in 3′ sequences.

The methods for selecting probes include finding polyadenylation sites and signals and select the probes from the region close to the polyadenylation sites, preferably within 300, 400, 500, 600 bases from the polyadenylation sites.

### EXAMPLE

Microarray gene expression chip design involves sequence selection, probe design, and feature design. This example shows various stage of a human gene expression chip design process employs embodiments of the methods of the invention to identify polyadenylation sites.

Table 1 shows the primary sequence and annotation information from a large variety of public databases (Table 1). Over six million sequences were considered for inclusion in this design.

TABLE 1
Sequence Sources

| Source | Release | Human sequences | In Unigene |
|---|---|---|---|
| UniGene | Apr. 20, 2001 (#133) | 2,688,626 | 7,907 |
| dbEST | Apr. 28, 2001 | 3,471,886 | 2,619,747 |
| WUSTL | February 2001 | 1,430,516* | 1,195,490* |
| GenBank | Apr. 25, 2001 | 61,523 | 38,168 |
| | (#123.0) | | |
| RefSeq | Apr. 30, 2001 | 12,716 | 12,461 |
| Golden | April 2001 | | |
| Path | | | |
| Total | | 7,665,267 | 3,873,773 |

UniGene clusters were used as a starting point for the design process. The use of primary sequence sources provided better control over the regions used and provided access to additional annotation information such as sequence quality parameters from dbEST. Raw base call information, which enables better polyadenylation identification, was obtained for a substantial number of EST sequences from the Washington University (WUSTL). The draft assembly of the human genome from the University of California, Santa Cruz (Golden Path) was used to improve cDNA sequence annotations

The success of an array design is highly dependent on the quality of sequence information used. To provide the most complete starting information, cDNA sequence data were obtained from primary sequence sources: GenBank, RefSeq dbEST and Washington University (WUSTL) (Table 1). Sequence meta information such as descriptions and definitions, clone identifiers, library identifiers, read directions, CDS annotations, low quality base annotations, gene names, and gene products were extracted from the external data files in addition to the actual sequence.

All input sequences were aligned to the draft assembly of the human genome (April 2001 release). Only high quality regions of genome alignment were used to annotate and analyze the input sequences. The genomic alignments also confirmed sequence orientation and consensus splice sites for many sequences.

In an effort to improve consensus sequence quality, low quality EST sequence regions were identified and removed according to the following rules:

Sequences were trimmed if the primary sequence annotation indicated poor quality regions.

If EST sequences aligned to the genomic sequence, the unaligned bases were removed.

The 3′ ends were trimmed in cases were the sequence read was abnormally long.

These approaches reduced the presence of low quality bases, which were shown to disrupt the clustering process and potentially contaminate the sequence content on the array.

Polyadenylation sites. Great care was therefore taken to identify polyadenylation sites since probes are generally selected within 600 bp upstream of the site, because some sample preparation protocols produce 3′ biased nucleic acid samples. The use of untrimmed, primary sequence information helped significantly in this regard because poly-A or poly-T tracts are often removed prior to submission to public databases. Polyadenylation sites were identified and a site score was calculated using a heuristic that accounts for the length of the poly-A (or poly-T, 5′ read), the amount of 5′ (or 3′) extraneous sequence, and the degree of interruption within the poly-A (or poly-T). For those sequences with a polyadenylation site, the presence of a polyadenylation signal was determined using a probabilistic model.

Vector contamination and repeats. Each sequence was assessed for repeats using RepeatMasker software and for vector contamination using BLASTN and the UniVector database.

Cluster Creation. The initial cluster information was derived from U133. Additional potential full length sequences not in UniGene were used to create an additional 1,144 singleton clusters.

Genome Based Subclustering. In a number of cases a UniGene cluster represents several genes within a gene family. Genome based subclustering was applied using the alignment information for each member sequence to the genomic sequence. Sequences that aligned to different contigs were assigned to separate subclusters. Those sequences that did not align to the genomic sequence were added to the largest subcluster.

Sequence Based Subclustering. At this time, the human genome assembly remains incomplete and the quality is highly variable. It is therefore still necessary to refine seed clusters using a transcriptome based clustering approach. This was accomplished using the Cluster and Alignment Tool (CAT). To be conservative in selecting probes, 75 percent identity in all of the member sequences is required when a consensus is called. This eliminates problems with ambiguous and polymorphic bases.

Orientation Based Subclustering. Subcluster orientation was determined using information from the following:

Sequence-label information; such as CDS annotations and read directions are used in the determination. In cases where introns are clearly delineated, consensus splice-site flanking sequences are used for orientation determination.

The intron flanking sequence GT-AG indicates the sense orientation while CT-AC implies an anti-sense orientation.

Polyadenylation signals and sites (5′ stretches of T's or 3′ stretches of A's) also provided orientation information.

A combination of the above information is used to make an orientation call of sense, anti-sense, or unknown for each member sequence used. Clusters with a problematic orientation were resubclustered by placing all the sequence members with evidence of a sense orientation into one subcluster and all the members with evidence of an anti-sense orientation into another subcluster. Sequences with an unknown orientation were placed into the larger of these.

Probe Selection Regions. A given subcluster, while typically representing one transcript variant, may represent several alternative polyadenylation sites that may be sufficiently spaced to warrant more than one probe selection region. Based on the orientation call, the 3′ end of the cluster is identified. For clusters of unknown or ambiguous orientation, probes were picked against both ends of the sequence. Potential transcript ends are identified by the 3′ end of a potential full length member sequence, by a set of 8 or more EST ends (5′ end of a 3′ EST or a polyadenylated EST), or by the end of the consensus sequence (FIG. 2). A 600 base region upstream of the end is chosen for probe selection. For putative transcript ends based on a potential full length mRNA, the corresponding mRNA sequence is used as an exemplar when picking probes. For all other transcript ends, the consensus sequence is used. A consequence of this strategy is that there can be multiple probe sets representing a particular sequence.

Prioritization. The number of potential regions to represent the human transcriptome and to pick probes from is in the hundreds of thousands. A number of these regions or clusters are speculative, consisting of EST singletons or aberrant minority subclusters resulting from cloning or sequencing errors. At the other extreme are transcripts that have been documented in the databases over 1000 times. Thus the set of potential regions for probes must be prioritized so that well-annotated and strongly supported regions are given the highest likelihood of being represented on the array. In general, the highest priority was given to regions representing mRNAs annotated as containing the complete coding region and some 3′ untranslated sequence. EST-only clusters are prioritized according to maximal depth of sequence alignment, a strong poly-adenylation site, the number of 3′ ends aligned with the poly-adenylation site, genomic mapping and orientation information. The actual prioritization rules are summarized in Table 2, below.

TABLE 2
Sequences on HG-U133

| | HG- | HG- | |
|---|---|---|---|
| Classification | U133A | U133B | Total |
| UniGene Clusters | 14,564 | 19,300 | 31,746 |
| Additional Potential Full | 527 | 2204 | 727 |
| lengths | | | |
| Subclusters | 18,605 | 21,099 | 39,092 |
| Full Length Including UTR | 13,187 | 1,574 | 14,685 |
| Extended Full Length- | 172 | 58 | 230 |
| Strongest evidence for | 3,236 | 6,934 | 10,153 |
| polyadenylation | | | |
| Complete CDS Consensus | 580 | 76 | 655 |
| End | | | |
| Non-EST Consensus End | 2,538 | 2,760 | 5,295 |
| Evidence for | 994 | 595 | 1,587 |
| polyadenylation | | | |
| EST-only clusters | | | |
| Oriented, Mapped, and 3p | 179 | 9,153 | 10,432 |
| Oriented and 3p | 33 | 590 | 623 |
| Mapped and 3p | 14 | 76 | 90 |
| 3p only | 0 | 22 | 22 |
| Opposite Consensus End | 683 | 619 | 1,301 |
| Distant Consensus End | 176 | 150 | 326 |

Table 2 Classifications and counts of sequences placed on the HG-U133 Set. It is estimated that the HG-U133 Set will interrogate over 31,000 genes and approximately 39,000 transcripts.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

What is claimed is:

1. A method for predicting a polyadenylation site comprising:
inputting a plurality of RNA transcript sequences or sequences dervied from RNA transcript sequences, wherein at least one sequence has its poly A or poly T tract sequence;
searching for a polyadenylation site, wherein the polyadenylation is an adenine rich region at the end of the sequence or a thymine rich region at the beginning of the sequence;
detecting the presence of polyadenylation signals neigboring the polyadenylation site by scanning the EST or RNA sequences or their corresponding genomic DNA sequences.

2. The method of claim 1 wherein the step of searching for a polyadenylation site comprising scanning the sequences for adenine rich region at the end of the sequence or a thymine rich region at the begining of the sequence.

3. The method of claim 2 wherein the adenine rich region comprises adenine in at least 50% of the region and the thymine rich region comprises thymine in at least 50% of the region.

4. The method of claim 2 wherein the adenine rich region comprises adenine in at least 60% of the region and the thymine rich region comprises thymine in at least 60% of the region.

5. The method of claim 2 wherein the adenine rich region comprises adenine in at least 70% of the region and the thymine rich region comprises thymine in at least 70% of the region.

6. The method of claim 2 wherein the adenine rich region comprises adenine in at least 80% of the region and the thymine rich region comprises thymine in at least 80% of the region.

7. The method of claim 1 wherein a heuristic score n_{A}/(n_{A}+0.5*(max(n_{R}−20,0))) is used for detecting adenine or thymine rich region; wherein n_{A} is the number of adenines or thymines in the block, and n_{R} is the number of bases after the block of adenines or thymine to the end of the sequence.

8. A method for detecting polyadenylation signal in a sequence with a polyadenylation site comprising searching for a polyadenylation signal hexamer in the sequence before the polyadenylation.

9. The method of claim 8 wherein the searching comprises evaluating the probability that there is a polyadenylation site: Pr(h=k|x) for k=6,7, . . . ,N, wherein the sequence before the polyadenylation site is x=(x₁,x₂, . . . x_{N}) and where x_{N} is the 3′-most base before the polyadenylation site.

10. The method of claim 9 wherein: Pr(h=k|x)=Pr(x|h=k) Pr(h=k) Pr(x).

11. The method of claim 10 wherein Pr(h=k|x)=Pr(xₖ₋₅, . . . ,xₖ|h=k) Pr(h=k)/Pr(xₖ₋₅, . . . ,xₖ) and wherein Pr(h=k) is the probability that the polyadenylation hexamer is located at position k in the sequence, at a distance (N−k) from the polyadenylation site, Pr(xₖ₋₅, . . . ,xₖ|h=k) is the probability of observing the hexamer (xₖ₋₅, . . . ,xₖ) given that it is a polyadenylation signal and Pr(xₖ₋₅, . . . ,xₖ|h≠k) is the probability of observing the hexamer given that it is not from a polyadenylation signal.

12. The method of claim 11 wherein the step of detecting comprises using a gamma function to produce a density which places the majority of its weight on the positions located 5 to 25 bases distant from the polyadenylation site.

13. The method of claim 12 wherein Pr(xₖ₋₅, . . . ,xₖ|h≠k), the probability of observing the hexamer given that it is not from a polyadenylation signal, is modeled using a second-order Markov model trained on data collected from human 3′ UTRs.

14. The method of claim 13 wherein Pr(xₖ₋₅, . . . ,xₖ|h≠k)=Pr(xₖ₋₅) Pr(xₖ₋₄|xₖ₋₅) Pr(xₖ₋₃|xₖ₋₅, xₖ₋₄) Pr(xₖ₋₂|xₖ₋₄,xₖ₋₃) Pr(xₖ₋₁|xₖ₋₃,xₖ₋₂) Pr(xₖ|xₖ₋₂,xₖ₋₁), wherein the first term is zero-order Markovian probability, the second is a first-order Markovian probability and the remaining four terms are second-order Markovian probabilities.

15. The method of claim 14 wherein, for a k^{th}-order Markov model, the probability of base b following a word w of length k is estimated by the frequency of the concatenated word (wb) divided by the frequency of the word w, where frequencies are computed from the training dataset of 3′UTR sequences.

16. The method of claim 15 wherein, for the case k=0 (a zero-order Markovian model), the probability of base b is estimated by its frequency in the dataset divided by the size of the dataset.

17. A computer readable medium comprising computer-executable instructions for performing the method comprising:
inputting a plurality of RNA transcript sequences or sequences dervied from RNA transcript sequences, wherein at least one sequence has its poly A or poly T tract sequence;
searching for a polyadenylation site, wherein the polyadenylation is an adenine rich region at the end of the sequence or a thymine rich region at the beginning of the sequence;
detecting the presence of polyadenylation signals neigboring the polyadenylation site by scanning the EST or RNA sequences or their corresponding genomic DNA sequences.

18. The computer readable medium of claim 17 wherein the step of searching for a polyadenylation site comprising scanning the sequences for adenine rich region at the end of the sequence or a thymine rich region at the begining of the sequence.

19. The computer readable medium of claim 18 wherein the adenine rich region comprises adenine in at least 50% of the region and the thymine rich region comprises thymine in at least 50% of the region.

20. The computer readable medium of claim 19 wherein the adenine rich region comprises adenine in at least 60% of the region and the thymine rich region comprises thymine in at least 60% of the region.

21. The computer readable medium of claim 20 wherein the adenine rich region comprises adenine in at least 70% of the region and the thymine rich region comprises thymine in at least 70% of the region.

22. The computer readable medium of claim 21 wherein the adenine rich region comprises adenine in at least 80% of the region and the thymine rich region comprises thymine in at least 80% of the region.

23. The computer readable medium of claim 17 wherein a heuristic score n_{A}/(n_{A}+0.5*(max(n_{R}−20,0))) is used for detecting adenine or thymine rich region; wherein n_{A} is the number of adenines or thymines in the block, and n_{R} is the number of bases after the block of adenines or thymine to the end of the sequence.

24. A computer readable medium comprising computer-executable instructions for performing the method comprising: searching for a polyadenylation signal hexamer in the sequence before the polyadenylation.

25. The computer readable medium of claim 24 wherein the searching comprises evaluating the probability that there is a polyadenylation site: Pr(h=k|x) for k=6,7, . . . ,N, wherein the sequence before the polyadenylation site is x=(x₁,x₂, . . .x_{N}) and where x_{N} is the 3′-most base before the polyadenylation site.

26. The computer readable medium of claim 25 wherein: Pr(h=k|x)=Pr(x|h=k) Pr(h=k)/Pr(x).

27. The computer readable medium of claim 26 wherein: Pr(h=k|x)=Pr(xₖ₋₅, . . . ,xₖ|h=k) Pr(h=k)/Pr(xₖ₋₅, . . . ,xₖ) and wherein Pr(h=k) is the probability that the polyadenylation hexamer is located at position k in the sequence, at a distance (N−k) from the polyadenylation site, Pr(xₖ₋₅, . . . ,xₖ|h=k) is the probability of observing the hexamer (xₖ₋₅, . . . ,xₖ) given that it is a polyadenylation signal and Pr(xₖ₋₅, . . . ,xₖ|h≠k) is the probability of observing the hexamer given that it is not from a polyadenylation signal.

28. The computer readable medium of claim 27 wherein the step of detecting comprises using a gamma function to produce a density which places the majority of its weight on the positions located 5 to 25 bases distant from the polyadenylation site.

29. The computer readable medium of claim 28 wherein Pr(xₖ₋₅, . . . ,xₖ|h≠k), the probability of observing the hexamer given that it is not from a polyadenylation signal, is modeled using a second-order Markov model trained on data collected from human 3′ UTRs.

30. The computer readable medium of claim 29 wherein Pr(xₖ₋₅, . . . ,xₖ|h≠k)=Pr(xₖ₋₅) Pr(xₖ₋₄|xₖ₋₅) Pr(xₖ₋₃|xₖ₋₅,xₖ₋₄) Pr(xₖ₋₂|xₖ₋₄,xₖ₋₃) Pr(xₖ₋₁|xₖ₋₃,xₖ₋₂) Pr(xₖ|xₖ₋₂,xₖ₋₁), wherein the first term is a zero-order Markovian probability, the second is a first-order Markovian probability and the remaining four terms are second-order Markovian probabilities.

31. The computer readable medium of claim 30 wherein, for a k^{th}-order Markov model, the probability of base b following a word w of length k is estimated by the frequency of the concatenated word (wb) divided by the frequency of the word w, where frequencies are computed from the training dataset of 3′UTR sequences.

32. The computer readable medium of claim 31 wherein, for the case k=0 (a zero-order Markovian model), the probability of base b is estimated by its frequency in the dataset divided by the size of the dataset.

33. A system comprising: a processor; and a memory coupled with the processor, the memory storing a plurality of machine instructions that cause the processor to perform logical steps of the method comprising:
inputting a plurality of RNA transcript sequences or sequences dervied from RNA transcript sequences, wherein at least one sequence has its poly A or poly T tract sequence;
searching for a polyadenylation site, wherein the polyadenylation is an adenine rich region at the end of the sequence or a thymine rich region at the beginning of the sequence;
detecting the presence of polyadenylation signals neigboring the polyadenylation site by scanning the EST or RNA sequences or their corresponding genomic DNA sequences.

34. The system of claim 33 wherein the step of searching for a polyadenylation site comprising scanning the sequences for adenine rich region at the end of the sequence or a thymine rich region at the begining of the sequence.

35. The system of claim 34 wherein the adenine rich region comprises adenine in at least 50% of the region and the thymine rich region comprises thymine in at least 50% of the region.

36. The system of claim 35 wherein the adenine rich region comprises adenine in at least 60% of the region and the thymine rich region comprises thymine in at least 60% of the region.

37. The system of claim 36 wherein the adenine rich region comprises adenine in at least 70% of the region and the thymine rich region comprises thymine in at least 70% of the region.

38. The system of claim 37 wherein the adenine rich region comprises adenine in at least 80% of the region and the thymine rich region comprises thymine in at least 80% of the region.

39. The system of claim 33 wherein a heuristic score n_{A}/(n_{A}+0.5*(max(n_{R}−20,0))) is used for detecting adenine or thymine rich region; wherein: n_{A} is the number of adenines or thymines in the block, and n_{R} is the number of bases after the block of adenines or thymine to the end of the sequence.

40. A system comprising a processor; and a memory coupled with the processor, the memory storing a plurality of machine instructions that cause the processor to perform logical steps of the method for detecting polyadenylation signal in a sequence with a polyadenylation site comprising: searching for a polyadenylation signal hexamer in the sequence before the polyadenylation.

41. The system of claim 40 wherein the searching comprises evaluating the probability that there is a polyadenylation site: Pr(h=k|x) for k=6,7, . . . ,N, wherein the sequence before the polyadenylation site is x=(x₁,x₂, . . . x_{N}) and where x_{N} is the 3′-most base before the polyadenylation site.

42. The system of claim 41 wherein: Pr(h=k|x)=Pr(x|h=k) Pr(h=k)/Pr(x).

43. The system of claim 42 wherein Pr(h=k|x)=Pr(xₖ₋₅, . . . ,xₖh=k) Pr(h=k)/Pr(xₖ₋₅, . . . ,xₖ) and wherein Pr(h=k) is the probability that the polyadenylation hexamer is located at position k in the sequence, at a distance (N−k) from the polyadenylation site, Pr(xₖ₋₅, . . . ,xₖ|h=k) is the probability of observing the hexamer (xₖ₋₅, . . . ,xₖ) given that it is a polyadenylation signal and Pr(xₖ₋₅, . . . ,xₖ|h≠k) is the probability of observing the hexamer given that it is not from a polyadenylation signal.

44. The system of claim 43 wherein the step of detecting comprises using a gamma function to produce a density which places the majority of its weight on the positions located 5 to 25 bases distant from the polyadenylation site.

45. The system of claim 44 wherein Pr(xₖ₋₅, . . . ,xₖ|h≠k), the probability of observing the hexamer given that it is not from a polyadenylation signal, is modeled using a second-order Markov model trained on data collected from human 3′ UTRs.

46. The system of claim 45 wherein Pr(xₖ₋₅, . . . ,xₖ|h≠k)=Pr(xₖ₋₅) Pr(xₖ₋₄|xₖ₋₅) Pr(xₖ₋₃|xₖ₋₅,xₖ₋₄)Pr(xₖ₋₂|xₖ₋₄,xₖ₋₃) Pr(xₖ₋₁|xₖ₋₃,xₖ₋₂) Pr(xₖ|xₖ₋₂,xₖ₋₁), wherein the first term is a zero-order Markovian probability, the second is a first-order Markovian probability and the remaining four terms are second-order Markovian probabilities.

47. The system of claim 46 wherein, for a k^{th}-order Markov model, the probability of base b following a word w of length k is estimated by the frequency of the concatenated word (wb) divided by the frequency of the word w, where frequencies are computed from the training dataset of 3′UTR sequences.

48. The system of claim 47 wherein, for the case k=0 (a zero-order Markovian model), the probability of base b is estimated by its frequency in the dataset divided by the size of the dataset.
